(19) United States
(12) Patent Application Publication
(10) Pub. No.: US 2010/0049155 A1
(43) Pub. Date: Feb. 25, 2010

(54) ABSORBENT GARMENT AND METHOD FOR MAKING IT

(75) Inventors: Anders Söderbergh, Savedalen (SE); Stefan Nedestam, Molnlycke (SE); Lucas Bäck, Billdal (SE)

Correspondence Address: BUCHANAN, INGERSOLL & ROONEY PC, POST OFFICE BOX 1404, ALEXANDRIA, VA, 22313-1404, US

(73) Assignee: SCA HYGIENE PRODUCTS AB, Goteborg (SE)

(21) Appl. No.: 12/443,716

(22) PCT Filed: Oct. 3, 2006
(86) PCT No.: PCT/SE06/01120

§ 371 Date: Mar. 31, 2009

(51) Int. Cl. A61F 13/51; A61F 13/45; A61F 13/64; B32B 38/04

(52) U.S. Cl. 604/385.23; 604/385.3; 604/392; 156/256

(57) ABSTRACT

Absorbent garment includes a containment assembly (8) having first and second transverse end edges (8a, 8b). The containment assembly includes a liquid pervious inner coversheet (6) on a wearer facing side, a liquid impervious outer coversheet (7) and an absorbent core (5) positioned between the inner and outer coversheets. The garment further includes at least one waist panel (9, 10) joined to one of the transverse end edges of the containment assembly (8), said waist panel (9, 10) is joined to the containment assembly (8) along a seam (11, 12) in which the wearer facing side of the waist panel faces the inner coversheet (6) of the containment assembly. The garment may be packed and stored folded in transverse direction (x) along said seam (11, 12). The invention also relates to methods for making the garment.

### TECHNICAL FIELD

The present invention relates to an absorbent garment such as a diaper, a pant diaper, an incontinence guard, a sanitary pant comprising a containment assembly and a waist panel joined to the containment assembly.

### BACKGROUND OF THE INVENTION

Absorbent garments of the above kind comprise a containment assembly having an absorbent core disposed between an inner coversheet and an outer coversheet. The containment assembly has a front portion intended to extend over the stomach of the wearer, a rear portion extending over the back of the wearer and a crotch portion there between. The containment assembly has a front and a rear transverse end edge. A waist panel is joined to at least one transverse end edge of the containment assembly and provides for an improved fit and comfort in the waist area. The waist panel may be elastic.

WO 96/18367 discloses an absorbent garment of this type. The garment comprises a containment assembly and fit panels joined to the respective transverse end edges of the containment assembly. The fit panel is elastically extensible in the longitudinal direction of the garment. A waist feature is joined to the waist edge of the fit panel, said waist feature is elastically extensible in lateral direction of the garment. A fastening system is disposed on the waist feature.

WO95/09594 discloses a sanitary or incontinence panty comprising a containment assembly which is elasticized along the crotch region. Elastic waistbands which are elastically extensible in transverse direction are joined to the respective transverse end edges of the containment assembly.

Absorbent garments are disposable and discarded after one single use. They are manufactured in high speed processes. Assembling of the different portions of the garment has to be made in a simple and cost efficient manner and be adapted to a high speed production line. At the same time the quality and function of the garment with respect to fit, comfort and capability to absorb and contain body fluids must be maintained.

### OBJECT AND FEATURES OF THE INVENTION

The present invention aims at providing an absorbent garment which solves the problem of providing high comfort and fit to a variety of body dimensions and which can be made with a cost-efficient manufacturing process at high production speed. This has according to certain embodiments of the present invention been accomplished by the fact that said waist panel is joined to the containment assembly along a seam in which the wearer facing side of the waist panel faces the wearer facing side of the containment assembly.

In one embodiment, the garment comprises first and second waist panels joined to the first and second transverse end edges respectively, of the containment assembly along first and second seams in which the wearer facing side of the respective waist panel faces the wearer facing side of the containment assembly.

In a further aspect, first fastening means for fastening together said garment to a pant-like shape are arranged at lateral sides of the first waist panel. Having fastening means attached to the waist panel, especially if this is of an elastic material, means that the garment may be tightened and fastened around the waist of the wearer with an improved fit and comfort.

In a still further embodiment of the invention, the garment is provided with opposed laterally extending belt members attached to lateral sides of the second waist panel, said belt members being adapted to be wrapped around the waist of the wearer of the garment and be fastened together by means of second fastening means, said belt members having a wearer facing inner side and an outer side.

In a belted garment, the first fastening means is adapted to be fastened to the outer side of the belt members, in such a way that the garment will assume a pant-like shape.

In an alternative embodiment, the garment is a pant-type absorbent article in which at least said first and second waist panels are joined to each other along their respective lateral side edges to form side seams.

According to one embodiment, the waist panel is elastic. In a further embodiment of the invention, said elastic waist panel is elastically extensible in at least the transverse direction of the article and is joined to the containment assembly in an extended state. In a further embodiment, the elastic waist panel may be elastically extensible also in the longitudinal direction of the article.

According to one embodiment, the garment is packed and stored folded in transverse direction along the seams between the containment assembly and the respective waist panel.

Having the waist panel joined to the containment assembly in the manner described means that the waist panels are simultaneously joined to and folded over the containment assembly in a configuration suited for packaging the product. When the product is taken out of the package the waist panels are unfolded to extend longitudinally outside the containment assembly. The seam between the containment assembly and the waist panel will protrude on the external side of the garment and will thus not risk to irritate the skin of the wearer.

The invention further relates to a method for manufacturing an absorbent garment as described, said method comprises the steps of feeding a plurality of containment assembly blanks aligned in longitudinal direction, feeding a web material and cutting it into lengths, each length being adapted to form at least one waist panel, joining each of said cut lengths of web material to a containment assembly blank to at least one of its transverse edges in a manner facing the inner coversheet of the containment assembly blank and so that the cut length of web material overlaps part of the respective containment assembly blank.

In one embodiment, a cut length of web material is joined to each transverse edge of the containment assembly blank.

According to a further embodiment, the plurality of aligned containment assembly blanks are interconnected to form a continuous web of longitudinally aligned containment assembly blanks, that each of said cut lengths of web material are joined to the web of aligned containment assembly blanks along an intended cutting line between two adjacent containment assembly blanks in such a manner that the cut length of web material overlaps part of each of the two adjacent containment assembly blanks on their inner coversheet side, and that subsequently the web material and the web of aligned containment assembly blanks are cut along said imaginary juncture line.

In a further embodiment of the invention, said web material intended to form said waist panels is an elastic web material.

### BRIEF DESCRIPTION OF THE DRAWINGS

FIG. 1 shows a plan view of an absorbent garment in the form of a diaper according to one embodiment of the invention.

FIG. 2 is a section according to the line II-II in FIG. 1.

FIG. 3 is a plan view of a belted garment according to an embodiment of the invention.

FIG. 4 is a simplified side view of the absorbent garment during unfolding after having been taken out from a package.

FIG. 5 shows a pant-type absorbent garment.

FIG. 6 shows schematically a part of a manufacturing line for making an absorbent garment according to an embodiment of the invention.

### DESCRIPTION OF PREFERRED EMBODIMENTS

The invention will be further described with reference to some embodiments shown in the drawings.

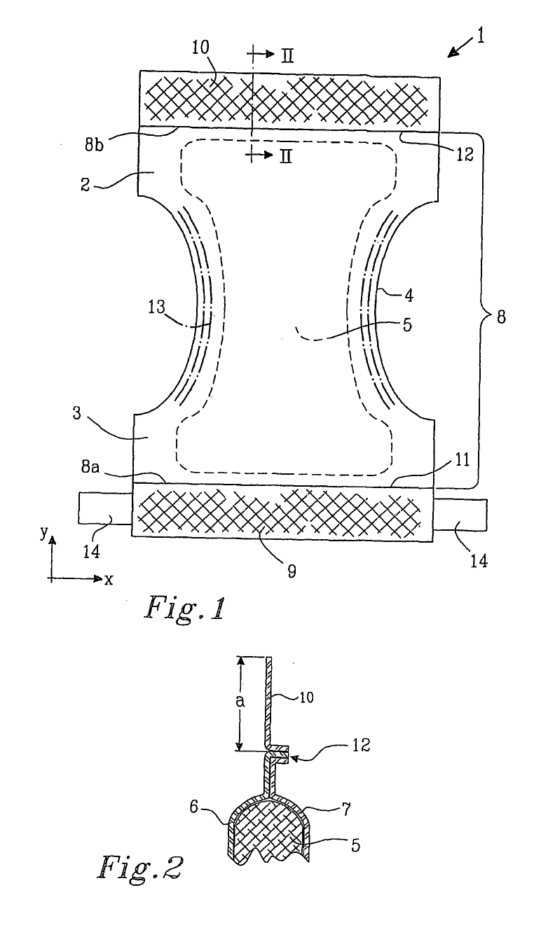

FIG. 1 shows an absorbent garment 1 in the form of a diaper having a longitudinal direction, y, and a transverse direction, x. The garment is shown from the wearer facing side in a flat uncontracted state and comprises a front portion 2 adapted to be applied over the stomach of the wearer, a rear portion 3 adapted to be applied over the back of the wearer and a crotch portion 4 adapted to extend over the crotch of the wearer between the legs. It is understood that the front and rear portions in some embodiments optionally may be used as either front or rear portions. An absorbent core 5 is disposed in the crotch portion 4 and extends into the front and rear portions 2 and 3. The absorbent core 5 is disposed between an inner coversheet 6 and an outer coversheet 7. The combined assembly of inner and outer coversheets and absorbent core is referred to as the containment assembly 8. The containment assembly has a pair of transverse end edges 8a and 8b.

The term “inner coversheet” refers to the liquid permeable material sheet forming the inner cover of the absorbent garment and which in use is placed in direct contact with the skin of the wearer. The inner coversheet can comprise a nonwoven material, e.g., spunbond, meltblown, carded, hydroentangled, wetlaid etc. Suitable nonwoven materials can be composed of natural fibers, such as wood pulp or cotton fibres, man-made fibres, such as polyester, polyethylene, polypropylene, viscose, rayon etc. or from a mixture of natural and man-made fibres. The inner coversheet material may further be composed of tow fibres, which may be bonded to each other in a bonding pattern, as, e.g., disclosed in EP-A-1 035 818. Further examples of inner coversheet materials are porous foams, apertured plastic films etc. The materials suited as inner coversheet materials should be soft and non-irritating to the skin and be readily penetrated by body fluid, e.g., urine or menstrual fluid. The inner coversheet may further be different in different parts of the absorbent garment.

The “outer coversheet” refers to the material forming the outer cover of the absorbent garment. The outer coversheet may be the same or different in different parts of the absorbent garment. At least in the area of the absorbent core the outer coversheet comprises a liquid impervious material, such as a thin plastic film, e.g., a polyethylene or polypropylene film, a nonwoven material coated with a liquid impervious material, a hydrophobic nonwoven material, which resists liquid penetration, or a laminate of a plastic film and a nonwoven material. The outer coversheet material may be breathable so as to allow vapour to escape from the absorbent core, while still preventing liquids from passing therethrough. Examples of breathable outer coversheet materials are porous polymeric films, nonwoven laminates of spunbond and meltblown layers and laminates of porous polymeric films and nonwoven materials. Preferably, the outer coversheet comprises a nonwoven material on at least the undergarment-facing surface thereof.

The “absorbent core” is the absorbent structure disposed between the two coversheets of the absorbent garment in at least the crotch region thereof. The absorbent core can be of any conventional kind. Examples of commonly occurring absorbent materials are cellulosic fluff pulp, tissue layers, highly absorbent polymers (so called superabsorbents), absorbent foam materials, absorbent nonwoven materials or the like. It is common to combine cellulosic fluff pulp with superabsorbent polymers in an absorbent core. Superabsorbent polymers are water-swellable, water-insoluble organic or inorganic materials capable of absorbing at least about 20 times their own weight of an aqueous solution containing 0.9 weight percent of sodium chloride. Organic materials suitable for use as superabsorbent materials can include natural materials such as polysaccharides, polypeptides and the like, as well as synthetic materials such as synthetic hydrogel polymers. Such hydrogel polymers include, for example, alkali metal salts of polyacrylic acids, polyacrylamides, polyvinyl alcohol, polyacrylates, polyacrylamides, polyvinyl pyridines, and the like. Other suitable polymers include hydrolyzed acrylonitrile grafted starch, acrylic acid grafted starch, and isobutylene maleic anhydride copolymers and mixtures thereof. The hydrogel polymers are preferably lightly cross-linked to render the material substantially water insoluble. Preferred superabsorbent materials are further surface cross-linked so that the outer surface or shell of the superabsorbent particle, fibre, flake, sphere, etc. possesses a higher crosslink density than the inner portion of the superabsorbent. The superabsorbent materials may be in any form which is suitable for use in absorbent composites including particles, fibres, flakes, spheres, and the like.

A high liquid storage capacity is provided by the use of high amounts of superabsorbent material. For an absorbent core comprising a matrix of hydrophilic fibres, such as cellulosic fibres, and superabsorbent material, the proportion of superabsorbent material is preferably between 10 and 90% by weight, more preferably between 30 and 70% by weight.

It is conventional for absorbent garments to have absorbent cores comprising layers of different properties with respect to liquid receiving capacity, liquid distribution capacity and storage capacity. The thin absorbent bodies, which are common in for example baby diapers and incontinence guards, often comprise a compressed, mixed or layered structure of cellulosic fluff pulp and superabsorbent polymers. The size and absorbent capacity of the absorbent core may be varied to suit different uses, such as infants or adult incontinent persons.

The absorbent core may further include an acquisition distribution layer placed on top of the primary absorbent body, which is adapted to quickly receive and temporarily store discharged liquid before it is absorbed by the primary absorbent core. Such acquisition distribution layers are well known in the art and may be composed of porous fibrous wadding or foam materials.

The garment 1 further comprises first and second waist panels 9, 10 joined to the respective transverse end edges 8a and 8b of the containment assembly 8 in an area where the inner and outer coversheets 6 and 7 extend longitudinally outside the absorbent core 5. The first and second waist panels are joined to the respective transverse end edge 8a and 8b of the containment assembly 8 along a seam 11 and 12 respectively, in which the wearer facing side of the waist panel faces the wearer facing side (the inner coversheet 6) of the containment assembly 8. This is illustrated in FIG. 2. The seam 11, 12 will thus protrude on the external side of the garment, while the inner wearer facing side of the garment in this area remains smooth. The waist panel is joined to the containment assembly 8 by any suitable method known in the art, such as by adhesive, ultrasonic welding, laser welding, thermobonding or the like.

In one embodiment, the waist panels 9, 10 are of an elastic material so that they are elastically extensible at least in the transverse direction, x. Their dimensions in transverse and longitudinal direction may vary depending on size of the article, but they preferably have a dimension in longitudinal direction of the article, y, of between 2 and 20 cm, preferably between 5 and 17 cm and more preferably between 8 and 12 cm. The dimension in longitudinal direction, y, of the garment is defined as the distance, a, between the seam 12 and the outer edge of the waist panel, which is illustrated in FIG. 2.

The elastic waist panels may be in the form of an elastic web material such as an elastic film, an elastic nonwoven, an elastic laminate or the like. The elastic laminate may be a laminate between two or more nonwoven layers, two or more film layers or a combination of film and nonwoven layers.

Examples of elastic laminates suitable for forming said elastic waist panels 9, 10 are any elastic laminate known in the art. One group of elastic laminates are so called “stretch-bonded” laminates, in which the elastic layer is stretched in at least one direction before laminating it with one or more inelastic layers. After the tension is removed from the elastic layer it can freely retract to its untensioned state, and the inelastic layer(s) laminated thereto become gathered, giving a three-dimensional puckering.

Another group of elastic laminates are so called “neck bonded” laminates, which refer to laminates in which an elastic material is bonded to a non-elastic material while the non-elastic member is extended under conditions reducing its width or necked. “Neck bonded laminate” refers to a composite material having at least two layers in which one layer is a necked, non-elastic layer and the other layer is an elastic layer. The layers are joined together when the non-elastic layer is in an extended condition.

A further group of elastic laminates are disclosed in for example WO/047488, in which inelastic nonwoven layers are laminated to an elastic film layer, and the laminate is stretched above the point of failure of the nonwoven materials, so that the inelastic layers break.

Examples of elastic laminates are described in EP-B-0 646 062, WO 98/29251, WO 03/000165 and U.S. Pat. No. 5,226,992. Examples of commercially available elastic laminates are FABRIFLEX 306 from Tredegar and PK 6358 from Nordenia.

In an alternative embodiment, the elastic waist panels 9, 10 comprise one or more elastic threads or strips contractably affixed between a pair of web materials, for example, nonwoven materials.

The elastic waist panels 9, 10 should have an elasticity in the transverse direction, x, of the garment of at least 30% as measured in the elasticity test as described below. They may also be elastically extensible in the longitudinal direction, y, of the garment.

The leg openings of the garment may be elasticized, said elastification is usually accomplished by a plurality of elastic members 13, such as elastic threads, which are contractably affixed between the outer and inner coversheets. The garment may also be provided by so called barrier cuffs, in order to provide an improved security against leakage. These barrier cuffs may in some instance replace leg elastics.

The garment is further provided with a pair of first fastening means 14 arranged at the opposite lateral edges of the first waist panel 9. The fastening means 14 may be an adhesive tape or mechanical fastening means especially hook-and-loop fastening means. Other types of mechanical fastening means include button and holes or button loops.

A “hook-and-loop fastener” refers to complementary fastening means having a “hook” portion and a “loop” portion and which are refastenable. The term “hook” as used herein refers to any element capable of engaging another element, the so called “loop” portion. The term “hook” is not limited to only “hooks” in its normal sense, but rather encompasses any form of engaging elements, whether unidirectional or bi-directional. The term “loop” is likewise not limited to “loops” in its normal sense, but also encompasses any structure capable of engaging with a “hook” fastener. Examples of “loop” materials are fibrous structures, like nonwoven materials. Hook-and-loop fasteners are for example available from Velcro, USA. The fibrous structures acting as “loop material” may for example form an external surface of the containment assembly, belt members and/or waist panels of the absorbent garment.

The first fastening means 14 is adapted to fasten to the opposite waist panel 10 either to the external surface thereof or to complementary fastening members. Having the first fastening means 14 attached to the waist panel 9, especially if these are of an elastic material, means that the garment may be tightened and fastened around the waist of the wearer with an improved fit and comfort.

In an alternative embodiment, the absorbent garment has only one waist panel, either at its front or rear portion 2 or 3.

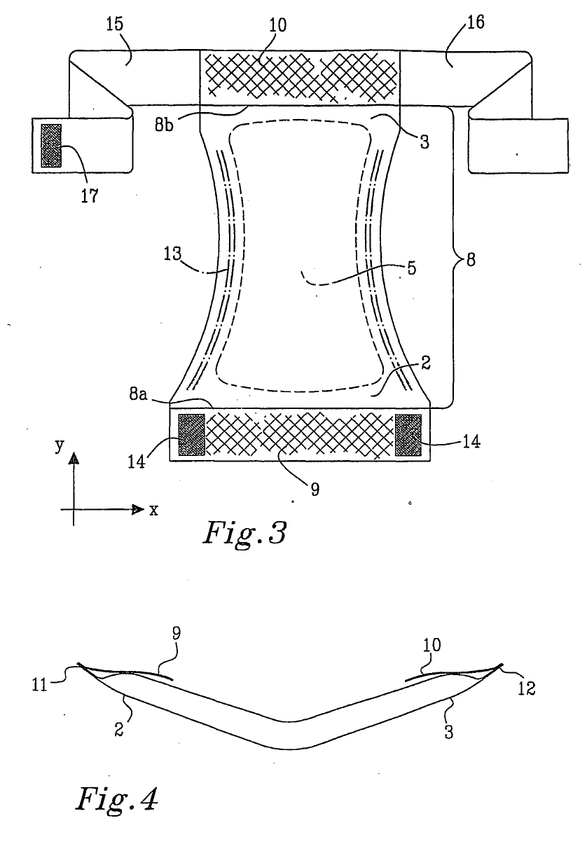

The absorbent garment shown in FIG. 3 further comprises a pair of opposed laterally extending belt members 15 and 16 attached to the second waist panel 10 intended to be applied on the back of the wearer. These belt portions 15 and 16 are adapted to be wrapped around the waist of the wearer of the garment and fastened together by means of second fastening means 17, such as mechanical fastening means, especially a hook member of a hook-and-loop fastener. The external surface of the opposite belt member, especially if this comprises a fibrous nonwoven, may function as a complementary loop member. Further examples of mechanical fasteners are button and holes or button loops, snap fasteners and the like. The buttons can either be fastened to the belt or to the garment. Alternatively the second fastening means 17 is an adhesive fastening means such as a tape tab, wherein the external surface of opposite belt member 16 may be of a material to which the tape can adhere.

The front part of the garment is then passed between the legs of the wearer and fastened to the outside or inside of the belt members by means of first fastening means 14 provided at the first waist panel 9. The garment will then assume a pant-like shape. Alternatively, the belt portions are arranged at the front waist panel 9 and the first fastening means 14 at the rear waist panel 10. Since often the front and back parts of the garment are more or less identical as to shape and size, the wearer or caretaker may apply the garment after individual preference, so that the front part may be used as rear part and vice versa.

The belt 15, 16 provides for improved possibilities to adjust the fit of the absorbent garment. It further simplifies the change of the garment especially when the wearer is standing up.

The width of the belt members 15, 16 may be from 5 to 20 cm, preferably from 8 to 15 cm and more preferably from 8 to 12 cm. The width is herein defined as the dimension of the belt members in the longitudinal direction, y, of the garment,

The belt members 15, 16 are for example made from a laminate of a carrier material, which forms the external surface of the belt, and a soft nonwoven, which forms the inside of the belt which is intended to be in direct contact with the skin of the user.

A suitable nonwoven material can be a spunbond material of, e.g., polypropylene or polyethylene fibres. Conjugate fibres may also be used. Another suitable nonwoven material is formed from a carded thermobonded material of, e.g., polypropylene, polyester or conjugate fibres.

The external surface of the belt members, for example the carrier material, can be adapted to function as a reception surface for the second fastening means 17. In case the fastening means is a hook fastener, a nonwoven material may be used as a carrier material. In case the fastening means 17 is a tape tab, a plastic film is suitable as carrier material, as for example described in WO 01/00129.

FIG. 4 is a simplified side view of an absorbent garment according to an embodiment of the invention in a position when it just has been taken out from a package and being unfolded from its folded state in the package. As can be seen the waist panels 9, 10 are folded over along the seams 11, 12 and overlaps the front and rear portions 2, 3 respectively of the containment assembly, and before use can be unfolded to form longitudinal extensions of the containment assembly.

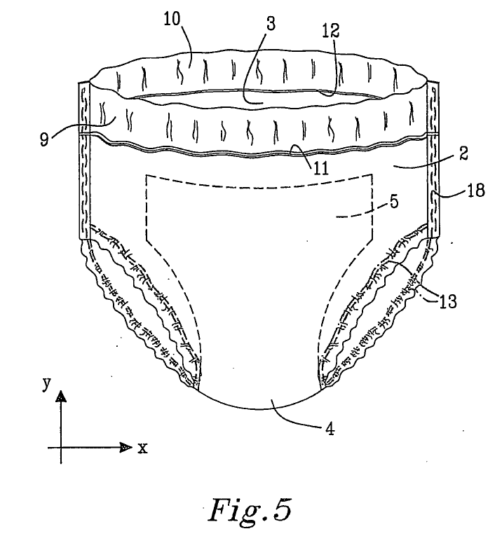

FIG. 5 shows a pant-type absorbent garment, such as a pant diaper, an incontinence pant, a sanitary pant or the like. The front and back portions 2 and 3 of the containment assembly are joined along their longitudinal side edges to form side seams 18. First and second waist panels 9 and 10 are joined to the transverse end edges of the front and back portions 2 and 3 in the manned described above and the waist panels are also joined along their longitudinal side edges to form extensions of said side seams 18. If the waist panels have sufficient extension in longitudinal direction, y, of the garment, it is sufficient to have only the waist panels joined along side seams 18.

It will be understood that the embodiments of absorbent garments described above and shown in the drawings are only examples and that the invention is applicable to any optional type of absorbent garment comprising a containment assembly with front and back panels applied against the stomach and back of the wearer and waist features for holding the garment around the waist of the wearer.

### Elasticity Test

The method measures how an elastic material behaves at repeated load and unload cycles. The sample is stretched to a predetermined elongation and a cyclic movement between 0 and said predetermined elongation is performed. Desired load and unload forces are recorded. The permanent, i.e. remaining, elongation of the relaxed material is measured.

A tensile tester, Lloyd LRX, able to perform cyclic movements and equipped with a printer/plotter or software presentation is used. The sample is prepared by cutting it to a width of 25 mm and a length that is preferably 20 mm longer than the distance between the clamps in the tensile tester. Marks for clamp distance are made in the sample in the non-elongated state.

The tensile tester is calibrated according to the apparatus instructions. The parameters needed for the test (load and unload forces) are adjusted to:

| | | |
|---|---|---|
| Crosshead speed: | 500 | mm/min |
| Clamp distance: | 50 | mm |
| Preload: | 0.05 | N |

The sample is placed in the clamps according to the marks and it is made sure that the sample is centered and fastened perpendicularly in the clamps. The tensile tester is started and three cycles between 0 and the predetermined elongation, equal to the highest defined 1^{st} load, are performed. Before the last cycle, the sample is relaxed for 1 minute, then the permanent elongation is measured by stretching the sample until a force of 0.1 N is detected and the elongation is read.

The permanent elongation after relaxation should be less than 10% and is measured by the method above. Thus an elasticity of 30% is defined as that the laminate should have a permanent relaxation after elongation of less than 10% after being exerted to an elongation of 30% in the tensile tester above. An elongation of 30% means an elongation to a length that is 30% longer than the initial length of the sample.

### Method for Manufacturing the Garment

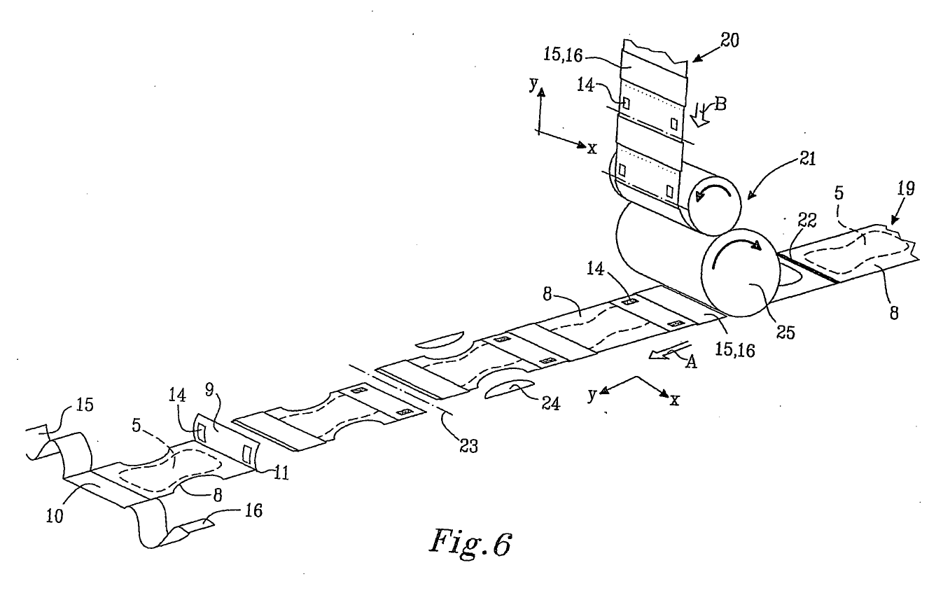

FIG. 6 illustrates schematically part of an exemplary manufacturing line for making an absorbent garment according to an embodiment of the invention, in this case a belted absorbent garment as shown in FIG. 3.

A continuous web 19 of longitudinally aligned containment assembly blanks 8 is forwarded in a first feeding direction A. The web of 19 containment assembly blanks 8 contains a plurality of absorbent cores 5 spaced apart a selected distance and applied between inner and outer coversheets 6 and 7 as described above. The web 19 is fed with the inner coversheet side facing upwards in the drawing. A web material 20 is fed in a second feeding direction B towards the web 19. In the embodiment shown in FIG. 6 the second feeding direction B is substantially transverse to the first feeding direction, but it is understood that the relationship between the feeding directions of the webs 19 and 20 may be optional. According to one embodiment, the web material 20 is an elastic web material.

The web material 20 has mounted thereto in spaced relationships belt members 15, 17 folded over the web 20 in transverse direction, and fastening members 14. Alternatively to folding the belt members, they may be rolled or unfolded. The web material 20 is fed into a unit 21, where it is cut into lengths, each length being adapted to form one or more waist panels 9, 10, which after cutting are slipped apart a selected distance. In the embodiment shown, these cut lengths have a length in longitudinal direction, y, corresponding to the length of two waist panels 9, 10. Each cut length comprises a pair of belt members 15, 16 and a pair of first fastening means 14. Subsequently to slipping apart the cut lengths may, provided that they are elastic, be stretched in transverse direction, x. However the stretching step is optional also for elastic materials.

After cutting, slipping apart and optional stretching of the cut lengths in transverse direction, x, they enter a press nip between a roll 25 and the web 19 of containment assembly blanks carried on a transport band or the like (not shown). During passage through the cutting, slipping apart and optional stretching unit 12 the feeding direction of the cut lengths of web material 20 is changed to be substantially in parallel with the feeding direction of the web 19 of containment assembly blanks. Before entering the press nip of the roll 25 a glue string 22 is applied transversely on the web 19 on the inner coversheet side 6 along an intended cutting line at approximately half the distance between two absorbent cores 5. The feeding of the cut lengths of web material 20 is registered with the feeding of the web 19 of containment assemblies, so that each cut length of web material 20 is centered over a glue string 22.

The glue string 22 has a sufficient dimension in longitudinal direction, y, so that the combined webs 19 and 20 may be cut in this area along said intended cutting line into individual garments each having a waist panel bonded thereto by the glue string. Alternatively two glue strings are applied, one on each side of the intended cutting line. The bonding of the cut lengths of waist material 20 to the web 19 of containment assembly blanks can of course be accomplished in other ways than by gluing, for example by thermobonding, ultrasonic welding, laser welding or the like.

The area of the cut lengths 9, 10 of web material 19 that is bonded to the inner coversheet side of the web 19 of containment assembly blanks will be referred to as the bonded area. In the areas outside the bonded area the cut lengths 9, 10 of web material 20 preferably are free from and not bonded to the web 19 of containment assembly blanks. A suitable dimension of the bonded area in longitudinal direction is between 4 and 30 mm, which after cutting and separation the combined material webs 19 and 20 will leave a bonded area between the waist panel 9, 10 and the respective containment assembly 8 of between 2 and 15 mm.

In the case the web material is an elastic material the cut lengths 9, 10 of web material 20 may optionally be kept in a stretched condition while being joined to the containment assembly. They preferably in such case, in their relaxed, unstretched condition, have a dimension in transverse direction, x, that is shorter than the corresponding transverse dimension of the web 19 of containment assembly blanks, while in the stretched condition they preferably, while joined to the web 19, have a transverse dimension substantially the same as the web 19.

The combined webs 19 and 20 are then cut at a cutting station 23 along the intended cutting line, which is located in the bonded area, into individual garments having a waist panel 9, 10 joined to the respective transverse end edges of the containment assembly 8. Cuts for the leg openings are also made in a further cutting station 24, if not made earlier in the process. The waist panels 9, 10 overlap part of the respective containment assembly 8 and the garment is preferably packaged in this state after folding along one or more further transverse folding lines.

After having been taken out from the package and before use of the garment the waist panels 9 and 10 are unfolded from the containment assembly 8 to form longitudinal extensions of the containment assembly 8 having the seam 12 between the waist panels and the containment assembly protruding on the outer coversheet side 7 of the garment, while the inner coversheet side 6 is smooth.

In alternative embodiments only one waist panel is joined to each containment assembly. The cut lengths of waist panel exiting the slip, cut- and stretch unit 21 may further correspond to the length in longitudinal direction, y, of one waist panel 9, 10. The containment assembly blanks 8 may also be separated from each other before being joined to the waist panel blanks.

The method described above provides for an increased production speed, since the main material web 19 of aligned containment assemblies has a shorter dividing distance than in conventional garments. The waist panels are combined with the web of containment assemblies from a separate material web and are applied to the web 19 overlaying it and thus not increasing the dividing distance of the aligned garment blanks of the combined webs 19 and 20. This means that more garments can be manufactured per minute with the same transport speed throughout the production line.

After cutting and separating the combined webs 19 and 20 into individual garments the garment is already in a folded condition with the waist panels 9, 10 being folded over the containment assembly 8, and the garment only needs to be folded for example along a central transverse folding line before being packaged.

It is understood that the process described above and shown in FIG. 6 can be modified in several ways within the scope of the claims and that additional components may be added to the material webs depending on the type of garment manufactured.

1. An absorbent garment, said garment having a longitudinal and a transverse direction and comprises a containment assembly having first and second transverse end edges, said containment assembly comprising a liquid pervious inner coversheet, a liquid impervious outer coversheet and an absorbent core positioned between the inner and outer coversheets, said garment further comprising at least one waist panel joined to one of the transverse end edges of the containment assembly said waist panel having an inner wearer facing side and an outer side, wherein said waist panel is joined to the containment assembly along a seam in which the wearer facing side of the waist panel faces the inner coversheet of the containment assembly.

2. The absorbent garment as claimed in claim 1, wherein the garment comprises first and second waist panels joined to the first and second transverse end edges respectively, of the containment assembly along said seam in which the wearer facing side of the respective waist panel faces the inner coversheet of the containment assembly.

3. The absorbent garment as claimed in claim 1, wherein first fastening means for fastening together said garment to a pant shape are arranged at lateral sides of the first waist panel.

4. The absorbent garment as claimed in claim 1, wherein the garment is provided with opposed laterally extending belt members attached to lateral sides of the second waist panel, said belt members being adapted to be wrapped around the waist of the wearer of the garment and be fastened together by means of second fastening means, said belt members having a wearer facing inner side and an outer side.

5. The absorbent garment as claimed in claim 3, wherein the first fastening means is adapted to be fastened to the outer side of the belt members, in such a way that the garment will assume a pant shape.

6. The absorbent garment according to claim 1, wherein the garment is a pant absorbent article in which at least said first and second waist panels are joined to each other along their respective lateral side edges to form side seams.

7. The absorbent garment as claimed in claim 1, wherein said at least one waist panel is an elastic waist panel.

8. The absorbent garment as claimed in claim 7, wherein said at least one elastic waist panel is elastically extensible in at least the transverse direction of the garment.

9. The absorbent garment as claimed in claim 8, wherein the elastic waist panel is joined to the containment assembly in an extended state.

10. The absorbent garment as claimed in claim 8, wherein said at least one elastic waist panel is elastically extensible also in the longitudinal direction of the garment.

11. The absorbent garment as claimed in claim 1, wherein said garment is packed and stored folded in transverse direction along said seams between the containment assembly and the respective waist panel.

12. A method for manufacturing an absorbent garment, said garment having a longitudinal and a transverse direction, wherein said garment comprises a containment assembly having first and second transverse end edges and comprises a liquid pervious inner coversheet on a wearer facing side, a liquid impervious outer coversheet and an absorbent core positioned between the inner and outer coversheets, comprising feeding a plurality of containment assembly blanks aligned in longitudinal direction, feeding a web material and cutting it into lengths, each length being adapted to form at least one waist panel, joining each of said cut lengths of web material to a containment assembly blank to at least one of its transverse end edges in a manner facing the inner coversheet of the containment assembly blank and so that the cut length of web material overlaps part of the respective containment assembly blank.

13. The method as claimed in claim 12, wherein a cut length of web material is joined to each transverse end edge of the containment assembly blank.

14. The method as claimed in claim 12 wherein the plurality of aligned containment assembly blanks are interconnected to form a continuous web of longitudinally aligned containment assembly blanks, said cut lengths of web material each have a length adapted to form two waist panels, each of said cut lengths of web material is joined to the web of aligned containment assembly blanks along an intended cutting line between two adjacent containment assembly blanks in such a manner that the cut length of web material overlaps part of each of the two adjacent containment assembly blanks on their inner coversheet side, and subsequently the web material and the web of aligned containment assembly blanks are cut along said intended cutting line.

15. The method as claimed in claim 12, wherein said web material intended to form said waist panels is an elastic web material.

16. The absorbent garment as claimed in claim 1, wherein the absorbent garment is a diaper, a pant diaper, an incontinence guard, or a sanitary pant.

17. The method as claimed in claim 1, wherein the absorbent garment is a diaper, a pant diaper, an incontinence guard, or a sanitary pant.
